(19) United States
(12) Patent Application Publication
(10) Pub. No.: US 2004/0048776 A1
(43) Pub. Date: Mar. 11, 2004

(54) Medicament for preventive and therapeutic treatment of fibrosis

(76) Inventors: Shin-ichi Satoh, Shizuoka (JP); Kazumi Shiraiwa, Shizuoka (JP)

Correspondence Address: BIRCH STEWART KOLASCH & BIRCH, PO BOX 747, FALLS CHURCH, VA, 22040-0747, US

(21) Appl. No.: 10/459,471

(22) Filed: Jun. 12, 2003

Related U.S. Application Data
(60) Provisional application No. 60/387,903, filed on Jun. 13, 2002.

(30) Foreign Application Priority Data
Jan. 31, 2001 (JP) 024512/2001

(51) Int. Cl. A61K 31/00

(52) U.S. Cl. 514/1

(57) ABSTRACT

A medicament for preventive and/or therapeutic treatment of fibrosis, which comprises, as an active ingredient, a substance selected from the group consisting of a compound represented by the following general formula (I) and a physiologically acceptable salt thereof, and a hydrate thereof and a solvate thereof:

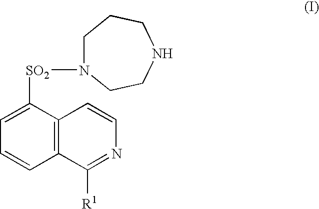

wherein R¹ represents hydrogen atom or hydroxy group.

### TECHNICAL FIELD

The present invention relates to a medicament for preventive and/or therapeutic treatment of fibrosis.

### BACKGROUND ART

Progression of fibrosis of tissues is observed in diseases such as nephritis, renal failure, hepatitis, hepatic cirrhosis or pulmonary fibrosis. Fibrosis of tissues is caused by the proliferation of interstitial cells such as a fibroblast as a typical example, and the generation of extracellular matrixes such as collagen. Excessive fibrosis induces sclerotic diseases of organs (Molecular, Medicine, Vol. 38, p. 854, Takehara et al., Senika to Shikkan (Fibrosis and Diseases)). At present, no effective medicament or no method for preventive or therapeutic treatment of fibrosis is available.

A compound represented by the following general formula (I) has an inhibitory activity against kinases such as Rho kinase, myosin light chain kinase or protein kinase C, and has a relaxing action on vascular smooth muscle, a blood flow increasing action, a blood pressure lowering action, a brain and heart protecting action and the like. The compound is known to be an effective substance in a vasodilator (particularly as a treating agent for angina pectoris), and a brain and heart protecting agent and the like (for example, Japanese Patent Unexamined Publication (Kokai) Nos. 61-152658, 61-227581, 2-256617, 4-264030, 6-056668, 6-080569 and 7-80854, WO98/06433, WO00/03746, Br. J. Pharmacol., 98, 1091 (1989), J. Pharmacol. Exp. Ther., 259, 738 (1991), Circulation, 96, 4357 (1997), Cardiovasc. Res., 43, 1029 (1999)).

### DISCLOSURE OF THE INVENTION

A medicament for preventive and/or therapeutic treatment of fibrosis has so far been desired to be provided.

The inventor of the present invention conducted intensive researches on the compound represented by the following formula (I). As a result, they have found that the compound has a preventive and/or therapeutic effect on fibrosis, which is totally unpredictable from the previously known actions such as the above-described vascular smooth muscle-relaxing action, blood flow increasing action, blood pressure lowering action, and brain and heart protecting action, and they achieved the present invention.

The present invention thus provides a medicament for preventive and/or therapeutic treatment of fibrosis, which comprises, as an active ingredient, a substance selected from the group consisting of a compound represented by the following general formula (I):

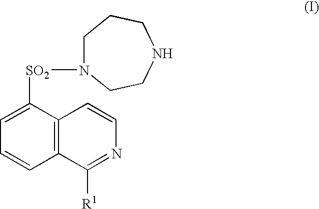

(wherein R¹ represents hydrogen atom or hydroxy group)and a physiologically acceptable salt thereof, and a hydrate thereof and a solvate thereof.

From another aspect, the present invention further provides the use of the substance selected from the group consisting of the compound represented by the above general formula (I)and a physiologically acceptable salt thereof, and a hydrate thereof and a solvate thereof, for manufacture of the aforementioned medicament; and a method for preventive and/or therapeutic treatment of fibrosis, which comprises a step of administering to mammals including humans, an preventively and therapeutically effective amount of the substance which is selected from the group consisting of the compound represented by the aforementioned general formula (I) and a physiologically acceptable salt thereof, and a hydrate thereof and a solvate thereof.

### BEST MODE FOR CARRYING OUT THE INVENTION

The compound represented by the general formula (I) of the present invention can be synthesized by known methods such as those described in Chem. Pharm. Bull., 40, (3) 770-773 (1992), and Japanese Patent Unexamined Publication (Kokai) No. 61-152658. As a salt thereof, an acid addition salt can be used, and for example, a pharmacologically acceptable nontoxic salt may preferably used. Examples of the salt include salts of inorganic acids such as hydrochloric acid, hydrobromic acid, phosphoric acid, and sulfuric acid, and salts of organic acids such as acetic acid, citric acid, tartaric acid, lactic acid, succinic acid, fumaric acid, maleic acid, and methanesulfonic acid.

When the medicament of the present invention is prepared as a pharmaceutical composition suitable for administration, the substance used as an active ingredient, which is selected from the group consisting of the compound represented by the aforementioned general formula (I) and a pharmacologically acceptable salt thereof, and a hydrate thereof and a solvate thereof may be mixed with a known pharmaceutically acceptable carrier.

Examples of the carrier include gelatin; sugars such as lactose and glucose; starches such as wheat, rice, corn; fatty acid such as stearic acid; fatty acid salts such as calcium stearate and magnesium stearate; talc; vegetable oil; alcohol such as stearic alcohol and benzyl alcohol; gum; and polyalkylene glycol. Further, examples of a liquid carrier generally include water, a physiological saline, a solution of dextrose or other similar sugar, and glycols such as ethylene glycol, propylene glycol, polyethylene glycol, and polypropylene glycol. When a capsule is prepared, it is generally preferred to use gelatin.

In a pharmaceutical composition containing the above-described carrier and the aforementioned substance as an active ingredient, the active ingredient is generally contained at 0.01 weight % or more and 80 weight % or less, and preferably 60 weight % or less based on the total weight of the composition.

The route of administration may be either oral or parenteral. Examples of formulations suitable for oral administration include tablets, capsules, powders, granules, liquids, and elixirs. An example of a preparation form suitable for parenteral administration includes a liquid preparation. When the pharmaceutical composition is parenterally administered as an intramuscular injection, intravenous injection, subcutaneous injection, drip infusion or the like, the composition is administered as a sterile solution added with other solutes such as sodium chloride or glucose, so that the pharmaceutical composition in the liquid form can be isotonic.

When the pharmaceutical composition is administered as an injection or drip infusion, it is also preferable to dissolve the active ingredient in sterilized water, a lidocaine hydrochloride solution (for intramuscular injection), physiological saline, glucose, a solution for intravenous injection, an electrolytic solution (for intravenous injection) or the like. When the pharmaceutical composition is prepared for a use as an injection, the active ingredient is generally contained at 0.01 weight % or more and 20 weight % or less based on the total weight of the composition. Preferably, the pharmaceutical composition can be prepared so that the active ingredient is contained at 0.1 weight % or more and 10 weight % or less based on the total weight of the composition. When the pharmaceutical composition is prepared in a liquid form for oral administration, preferred examples of the liquid form include a suspension or syrup, wherein the active ingredient is contained at 0.01 weight % or more and 20 weight % or less based on the total weight of the composition. Examples of a carrier of the aforementioned composition include aqueous excipients such as perfume, syrup, or a pharmaceutical micelle.

The medicament of the present invention has an action of suppressing the progression of fibrosis to protect organs, and/or an action of des-fibrosis of already-formed fibrous tissues. Therefore, the medicament can be used as a medicament for preventive and/or therapeutic treatment of fibrosis. Although it is not intended to be bound by any specific theory, it is known that the fibrosis of tissues is caused by a proliferation of interstitial cells including a fibroblast as a typical example, and a generation of extracellular matrixes such as collagen. It is considered that the medicament of the present invention can preventively and/or therapeutically treat fibrosis of tissues on the basis of the action of suppressing the proliferation of interstitial cells and/or the generation of extracellular matrixes. Types of diseases, that cause fibrosis to be treated by the medicament of the present invention, are not particularly limited. For example, fibrosis of tissues in diseases such as nephritis, renal failure, hepatitis, hepatic cirrhosis, or pulmonary fibrosis is a preferred target to be treated with the medicament of the present invention.

Doses of the medicament of the present invention are not particularly limited. A dose can be suitably selected depending on the age, health condition, body weight, and degree of symptoms of a subject to be administered, a kind or frequency of other simultaneous treatment if applied, a nature of a desired effect, an administration route, an administration protocol and the like. Generally, the dose may be 0.01 mg/kg or more and 20 mg/kg or less per day for parenteral administration and 0.02 mg/kg or more and 40 mg/kg or less per day for oral administration.

### EXAMPLES

The present invention will be explained more specifically below by referring to examples and reference examples. However, the scope of the present invention is not limited to the examples.

### Example 1

### Action on Rat Models with Unilateral Ureter Ligation

Rats with unilateral ureter ligation were used as models of the fibrosis of the kidney (Sommer M. et al., Nephron 82, 39-50, 1999; Sato N. et al., Nephron 89, 177-185, 2001).

The rats were anesthetized, and the ureters were ligated. Two weeks later, the kidneys were dissected out. Pathological specimens were prepared, and the fibrosis of the cortical part was measured by staining with Sirius Red. A compound represented by the general formula (I) (wherein R¹ is hydrogen atom; referred to as “Compound 1” hereinafter in examples) was dissolved in a physiological saline, and starting from the next day of the ureter ligation, the compound was intraperitoneally administered at a ratio of 3 and 10 mg/kg/day once a day for 2 weeks.

Two weeks after the ureter ligation, 2.50±0.38% (mean value±mean error; 7 cases) of the cortical part of the kidney was found to be fibrosed in the group of rats to which Compound I was not administered. Whilst administratin of Compound 1 gave suppression of the area of the fibrosis as low as 1.66±0.23% (3 mg/kg group; 7 animals) and 1.43±0.19% (10 mg/kg group; 7 animals; p<0.05, Dunnett's test).

### Example 2

### Acute Toxicity

The acute toxicity test of the compounds represented by the formula (I) (Compound 1; and a compound represented by the formula (1), wherein R¹ is hydroxyl group: hereinafter referred to as “Compound 2” in examples) was conducted by using rats (Jcl: Wistar, 5-week old) and mice (Slc:ddY, 5-week old). As a result, the compounds are verified to have low toxicity. The results are shown in Table 1.

TABLE 1

| | | Administration | | Result |
|---|---|---|---|---|
| Compound | Animal | route | Sexuality | LD₅₀ (mg/kg) |
| Compound 1 | Rat | Intravenous | Male | 59.9 |
| | | | Female | 63.9 |
| | | Oral | Male | 335.0 |
| | | | Female | 348.0 |
| | | Subcutaneous | Male | 123.2 |
| | | | Female | 128.3 |
| Compound 1 | Mouse | Intravenous | Male | 63.7 |
| Compound 2 | Mouse | Intravenous | Male | 119.3 |

### Example 3

Formulation Examples Sterile Injections

The ingredients shown in the following Table 2 were dissolved in distilled water for injection, and then the distilled water for injection was added up to the necessary final weight. Two ml of the solution was sealed in an ampoule followed by sterilization by heating.

TABLE 2

| | Ingredient | Amount |
|---|---|---|
| Formulation 10 mg | Compound 1 · hydrochloride | 10 mg |
| | Sodium chloride | 16 mg |
| | Distilled water | Appropriate amount |
| | | 2 ml in total volume |
| Formulation 30 mg | Compound 1 · hydrochloride | 30 mg |
| | Sodium chloride | 16 mg |
| | Distilled water | Appropriate amount |
| | | 2 ml in total volume |
| Formulation 60 mg | Compound 1 · hydrochloride | 60 mg |
| | Sodium chloride | 16 mg |
| | Distilled water | Appropriate amount |
| | | 2 ml in total volume |
| Formulation 10 mg | Compound 2 · hydrochloride | 10 mg |
| | Sodium chloride | 16 mg |
| | Distilled water | Appropriate amount |
| | | 2 ml in total volume |
| Formulation 30 mg | Compound 2 · hydrochloride | 30 mg |
| | Sodium chloride | 16 mg |
| | Distilled water | Appropriate amount |
| | | 2 ml in total volume |
| Formulation 60 mg | Compound 2 · hydrochloride | 60 mg |
| | Sodium chloride | 16 mg |
| | Distilled water | Appropriate amount |
| | | 2 ml in total volume |

### Example 4

### Formulation Examples Tablets

Tablets containing the ingredients shown in the following Table 3 were prepared by an ordinary method.

TABLE 3

| | Ingredient | Amount | |
|---|---|---|---|
| Formulation 10 mg | Compound 1 · hydrochloride | 10.0 | mg |
| | Crystalline cellulose | 25.0 | mg |
| | Lactose | 108.5 | mg |
| | Magnesium stearate | 1.5 | mg |
| | Carboxymethylcellulose calcium | 5.0 | mg |
| | Total | 150.0 | mg |
| Formulation 20 mg | Compound 1 · hydrochloride | 20.0 | mg |
| | Crystalline cellulose | 25.0 | mg |
| | Lactose | 98.5 | mg |
| | Magnesium stearate | 1.5 | mg |
| | Carboxymethylcellulose calcium | 5.0 | mg |
| | Total | 150.0 | mg |
| Formulation 10 mg | Compound 2 · hydrochloride | 10.0 | mg |
| | Crystalline cellulose | 25.0 | mg |
| | Lactose | 108.5 | mg |
| | Magnesium stearate | 1.5 | mg |
| | Carboxymethylcellulose calcium | 5.0 | mg |
| | Total | 150.0 | mg |
| Formulation 20 mg | Compound 2 · hydrochloride | 20.0 | mg |
| | Crystalline cellulose | 25.0 | mg |
| | Lactose | 98.5 | mg |
| | Magnesium stearate | 1.5 | mg |
| | Carboxymethylcellulose calcium | 5.0 | mg |
| | Total | 150.0 | mg |

What is claimed is:

1. A medicament for preventive and/or therapeutic treatment of fibrosis, which comprises, as an active ingredient, a substance selected from the group consisting of a compound represented by the following general formula (I) and a physiologically acceptable salt thereof, and a hydrate thereof and a solvate thereof: 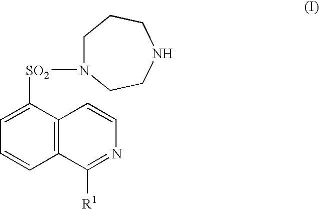
wherein R¹ represents hydrogen atom or hydroxy group,.

2. The medicament according to claim 1, wherein the fibrosis is resulting from nephritis or renal failure.

3. The medicament according to claim 1, wherein the fibrosis is resulting from hepatitis or hepatic cirrhosis.

4. The medicament according to claim 1, wherein the fibrosis is resulting from pulmonary fibrosis.
